(19) United States
(12) Patent Application Publication
(10) Pub. No.: US 2009/0136478 A1
(43) Pub. Date: May 28, 2009

(54) Methods for using enzymes to disintegrate cancerous tumors in vivo

(76) Inventor: Alon Shomer, Moshav Kefar Uriyya (IL)

Correspondence Address: Alon Shomer, Moshav Kefar Uriyya 35, D.N. Shimshon, Moshav Kefar Uriyya, 99735, IL

(21) Appl. No.: 12/292,583

(22) Filed: Nov. 21, 2008

Related U.S. Application Data
(60) Provisional application No. 61/004,083, filed on Nov. 26, 2007.

(51) Int. Cl. A61K 38/48; A61K 31/60; A61P 35/00

(52) U.S. Cl. 424/94.66; 514/159

(57) ABSTRACT

Methods to disintegrate cancerous tissue in vivo, using proteolytic enzyme compound for the digestion of the cancer cells. These methods exploit the fact that there is a significant difference in the strength and layout of the protein fraction between the normal and the cancer cells.

This application claims the benefit of U.S. Provisional Application No. 61/004,083 filed Nov. 26, 2007, which is incorporated herein by reference in its entirety.

### BACKGROUND

The morphological difference between normal and cancer cells is significant enough in many aspects to provide methods to interfere with the growth or to selectively destroy the cancer tissue.

As the cancer tissue in being destroyed there is an interest to minimize the interference or the damage that may take place to the normal tissue. The aim is to look for materials that affect the cancer tissue profoundly and at the same time will cause no or minimal damage to the normal tissue.

The treatments of cancer growths as of today are deficient in tools that will enable the following three characteristics: safe, quick acting and the specific destruction of cancer tissues with minimal damage to normal tissues.

The treatment presented here is marked very high in all the three parameters: safe, quick acting and specific, although it has the disadvantage of the need for direct contact with the cancer tissue.

It is based on the significant difference in the protein makeup of the cells. There is a significant difference between the cancerous and normal cells and tissues, in the quantity, strength and layout of the protein fractions.

Cancer malignant cells that grow very rapidly, lack or have many of its proteins poorly formed.

In cancer cells the skeletal proteins such as actine are deficient or poorly formed. The connection between the cytoplasm proteins such as actin and spectrin and the membrane proteins is absent or significantly reduced. This leads to a significant increase in the membrane protein's mobility, which is probably the reason for the reduction or absence of the protein's layout on the outer surface (such as fibronectin) of the cell and hence to the reduction of the cell to cell connection and a rounder cell shape. The increase in mobility of the cancer cell membrane proteins is also an advantage taken by this application.

The more malignant the cancerous cells are, the greater these morphological differences are.

### SUMMARY OF THE INVENTION

This invention is based on the use of proteolytic enzymes for the destruction of cancerous tumors in vivo with minimal damage to normal tissue, where the tumor is embedded. The invention sheds light and takes advantage of the vast difference in the sensitivity to the proteolytic action, between the normal and cancer tissue.

As a result of the absence, weakness and changed dynamics of the protein fractions that characterize the cancer cells, it becomes vulnerable to the digestion activity of some proteolytic enzymes.

The morphological differences between normal and cancer cells, while focusing on the vulnerability of the cancer cells to the proteolytic actions are:

A) A rounder shape of cell which is loosely connected to its neighboring cell makes it easier for the proteolytic enzymes to come in contact with cancerous cells.

B) A cell that is less covered—protected by outer surface proteins is more reachable by proteolytic enzymes.

C) Increased mobility of membrane proteins causes a significant increase in the interaction between its membrane proteins and available proteolytic enzymes.

D) Reduced cytoskeletal proteins make the cancerous cell more prone to proteolysis.

These differences between the cancer and normal cell enable some proteases to digest the unprotected cancer cell quickly, without significantly harming the normal cells around them.

These differences of sensitivity to the proteolytic enzymes between the two tissues enable the safe treatment suggested herein.

The end result of the proteolytic activity is a quick and complete destruction of the cancer cells while the normal tissue may be affected to an acceptable degree.

Some proteolytic enzymes show greater affinity to digest the cancer cells while affecting the normal cells to a much lesser degree.

Cancerous tumors are often covered by keratin.

To achieve the proteolytic destruction of the cancerous tissue which is still weak under the keratin, there is a need to remove the keratin.

Materials such as salicylic acid, lactic acid, urea or others are capable of removing or disintegrating such proteins as keratin and may be included when necessary as a part of the treatment.

The best proteolytic enzyme I found so far for this patent is pepsin with hydrochloric acid for maintaining an optimal pH.

### DETAILED DESCRIPTION OF THE INVENTION

There is a large volume of data in the scientific literature that describe the difference in the protein makeup, between normal and cancerous tissue as was elaborated here previously. This difference exposes a weakness in the cancer cells that can be exploited by using proteolytic enzymes.

The aim of the use of the proteolytic enzymes is to digest and breakdown the unprotected cancer cells quickly.

At the same time the normal tissue which in it the cancerous tumor is located should not be affected to a significant level. A significant destruction of the normal tissue, when digesting the cancerous tumor may render the whole treatment useless.

The specific amount and concentration of an active proteolytic enzyme needed to digest the tumor and cause no significant destruction of the normal tissue lies between the sensitivity of the tissues. This gap between sensitivity of the tissues differs depending on the type of normal and cancerous tissues.

The concentrations of the proteolytic enzyme of choice should be maintained with in this gap so only the cancerous tumor will break down.

The amount of enzyme acid compound is important since we desire that it will come in contact only with the cancerous tissue. Monitoring this, as much as possible, and allowing mostly the cancerous tumor to come in contact with the enzyme acid compound in its active form, will allow treatment in cases that the sensitivity gap is narrow.

In some cases such as open wounds the sensitivity gap may not exist, this treatment may also be used even though the normal tissue will be damaged. As the tumor is quickly and completely destroyed, the wound in the normal tissue may heal fast. Using the proteolytic enzyme in such cases may be weighed against other methods of practice.

After viewing several proteolytic enzymes, the protease that was found to be able to bring fast and complete disintegration of the cancer tissue is pepsin, when kept in optimum pH for its activity.

Pepsin with hydrochloric acid in optimal pH was found to be most effective in the destruction of the cancerous tissue. The best results are obtained when using pepsin natural companion: hydrochloric acid (HCl). Other acids may replace HCl in maintaining the optimal pH for its activity.

Pepsin with HCl are natural to many animals stomach and function in breaking down large variety of proteins, even tough ones as collagen.

The pepsin source suggested for the treatment can be from different sources, although porcine pepsin has been used successfully.

Fish pepsin of many species has a greater affinity for digesting also keratin, and may be the preferred choice when there is a need to remove keratin from the cancerous tumor. Other sources of pepsin may produce the desirable results.

Pepsin, depending on its source, has an optimum pH for maximum activity of 2-3.7.

The temperature of optimal activity of porcine source is 37-40 degrees C., where fish pepsin, depending on its environment, its optimal temperature is much lower.

A commercial products of pepsin and HCl are available as digestion aid.

Some of these products are suitable for use for the purpose of disintegrating cancerous tumors when mixed with some sterile water. It is then active.

The tumor may disintegrate on contact or may need to have the active compound rubbed into the tumor for the disintegration of the tumor.

Other proteolytic enzymes may be used to disintegrate cancerous tumors in vivo, when kept in their optimal pH and made active.

The protein keratin as well as some other proteins may interfere with the action of pepsin in breaking down the cancerous tumors since the concentration of pepsin that is needed to remove the keratin will cause normal tissue disintegration.

For this purpose, other materials such as salicylic acid, lactic acid, urea and others, that are relatively harmless to the body in the concentrations that are needed, may be used before or during the use of pepsin.

I claim:

1) A method to breakdown cancerous tissue in vivo, using a compound containing the proteolytic enzyme pepsin and hydrochloric acid.

2) The method in claim 1—wherein the concentration and quantity of the compound is kept so it will lead to breakdown of the cancerous tumor, and at the same time will cause minimal damage to the normal tissue surrounding the tumor.

3) The method in claim 1 wherein the proteolytic enzyme pepsin is substituted with another proteolytic enzyme and the hydrochloric acid is substituted with appropriate acid or alkaline to maintain the pH for enzyme activity.

4) The method in claim 1 wherein the hydrochloric acid is substituted with another acid.

5) A method to breakdown cancerous tissue protected by keratin in vivo using a compound containing salicylic acid to remove or dissolve the keratin around and/or within the tumor, with or without the proteolytic enzyme pepsin and hydrochloric acid.

6) The method in claim 5—wherein the concentration and quantity of the compound is kept so it will lead to breakdown of the cancerous tumor, and at the same time will cause minimal damage to the normal tissue surrounding the tumor.

7) The method in claim 5 wherein the proteolytic enzyme pepsin is substituted with another proteolytic enzyme and the hydrochloric acid is substituted with appropriate acid or alkaline to maintain the pH for enzyme activity.

8) The method in claim 5 wherein the hydrochloric acid is substituted with another acid.

9) The method in claim 5 wherein the salicylic acid is substituted with other materials such as urea or/and lactic acid.
